(19) United States
(12) Patent Application Publication
(10) Pub. No.: US 2013/0109991 A1
(43) Pub. Date: May 2, 2013

(54) Bad Breath Detection System

(71) Applicant: Michael McLain, Cabool, MO (US)

(72) Inventor: Michael McLain, Cabool, MO (US)

(21) Appl. No.: 13/281,549

(22) Filed: Oct. 26, 2011

(51) Int. Cl. A61B 5/08

(52) U.S. Cl. 600/530

(57) ABSTRACT

A bad breath detection system comprising: a bad breath detection device; and a indicating agent on the bad breath detection device wherein when a user places the bad breath detection device in their mouth the indicating agent provides a visual cue depending on a concentration of malodorous compounds in a saliva sample of the user to indicate to the user a level of breath freshness. The bad breath detection device may be embodied in various forms including a tube where the user blows into the tube for detection, a strip where an end of the strip is placed in the user's mouth or a disc where the user places the disc into their mouth for detection.

### BACKGROUND OF THE INVENTION

1. Field of the Invention

The present invention relates to an indicating device for detecting malodorous chemicals and compounds in the saliva and breath of the user.

2. Description of Related Art

Typically, people in today's society are very aware of their physical appearance and personal hygiene. Most people would never leave their house without combing their hair and brushing their teeth. While mirrors allow a person to check how they look throughout the day, it is more difficult for them to check their breath after brushing. Some people are very self-conscious of their breath especially after eating or mouth breathing for an extended period of time. They may carry gum or mints to mask any odors or keep a toothbrush and toothpaste with them to brush periodically during the day.

The methods to check one's breath are very limited. Some people may cup their hand over their mouth and nose and then exhale into their hand to smell any bad odors present. This method is not always reliable because much of the malodorous smells may escape or the person may not be able to smell their own breath. Alternatively, a person may lick their wrist to smell their saliva and try to detect any bad odors. This method usually fails because people do not know the difference between clean good smelling saliva and bad breath saliva. Plus, most people find it gross to lick themselves in an attempt to determine the freshness of their breath. Finally, the most intrusive, yet most reliable, way to check one's breath is to actually exhale into the face of another person. The other person may quickly assess whether or not the breath is fresh or offensive. Even facial expressions may be indicators of the person's breath.

It would beneficial in the art to provide a bad breath detector to visually indicate whether or not a person has bad breath. It would also be desirable to have a bad breath detector which is cheap and easy to use for a person on the go.

### SUMMARY OF THE INVENTION

In view of the foregoing disadvantages inherent in the prior art, the general purpose of the present invention is to provide a bad breath detection system, configured to include all of the advantages of the prior art, and to overcome the drawbacks inherent therein.

Accordingly, an object of the present invention is to provide a bad breath detection device as a visual indicator to determine the level of freshness of a user.

Another object of the present invention is to provide a bad breath detection device that is portable as a quick and easy method of fresh breath detection.

To achieve the above objects, in an aspect of the present invention, a bad breath detection system is described comprising: a bad breath detection device; and a indicating agent on the bad breath detection device wherein when a user places the bad breath detection device in their mouth the indicating agent provides a visual cue depending on a concentration of malodorous compounds in a saliva sample of the user to indicate to the user a level of breath freshness. The bad breath detection device may be embodied in various forms including a tube where the user blows into the tube for detection, a strip where an end of the strip is placed in the user's mouth or a disc where the user places the disc into their mouth for detection.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings, wherein like elements are identified with like symbols, and in which:

FIG. 1 is a perspective view of a breath detection tub in accordance with an exemplary embodiment of the present invention;

FIG. 2 is a perspective view of a breath detection strip in accordance with an alternative embodiment of the present invention; and

FIG. 3 is a perspective view of a breath detection disc in accordance with an alternative embodiment of the present invention.

### DETAILED DESCRIPTION OF THE DRAWINGS

The present invention relates to a bad breath detection system comprising a bad breath detection device, and an indicating agent on the bad breath detection device wherein when a user places the indicating agent in their mouth, and the indicating agent changes from a light color to a dark color depending on a concentration of malodorous compounds in a saliva sample of the user. Simply, the present invention is an indicating device for detecting malodorous chemicals and compounds in the saliva and breath of the user to indicate to the user their level of breath freshness.

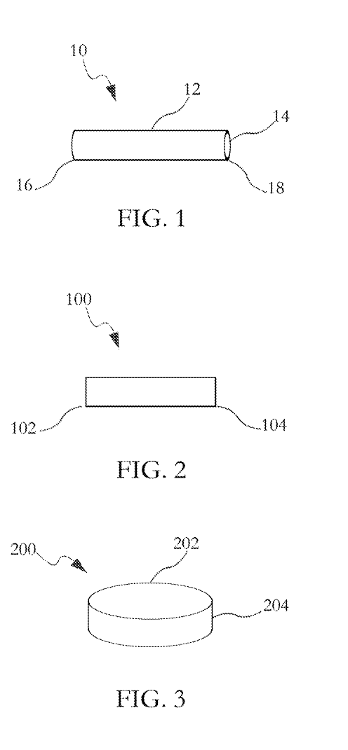

Turning now descriptively to the drawings, referring to FIG. 1, a perspective view of a bad breath detection tube (10) is shown in accordance to an exemplary embodiment of the present invention. The bad breath detection tube (10) may comprise an elongated body (12) with a mouthpiece (18) at a first end of the elongated body (12) and an evacuation point (16) at an opposing end. The mouthpiece (18) has a hollow opening (14) where a user blows into the bad breath detection tube (10). The evacuation point (16) may be partially open to allow an exhale of breath to escape the elongated body (10). The evacuation point (16) may include an indicating agent to detect a malodorous chemical or compound in the exhale of breath. The bad breath detection tube (10) may be disposable to enable the user to throw the device away after use. Alternatively, the device may be reusable by allowing the indicating agent to be replaced between uses at the evacuation point (16).

The indicating agent may detect the acidic nature of the breath, the ammonia concentration, the sulfuric concentration or a variety of different chemicals present in the exhale of breath. Small amounts of saliva present in the user's breath are received on the indicating agent to determine the malodorous compounds. The indicating agent provides a visual cue like changing color depending on the acidity or chemical concentration of malodorous compounds present in the saliva. The color change becomes more extreme as the malodorous concentration increases. Thus, if low concentrations of malodorous compounds are present the color of the indicating agent may be light whereas if the concentration of malodorous compounds is high the color of the indicating agent may be dark. Likewise, specific colors may indicate a specific concentration, similar to how litmus paper works to indicate pH levels.

Referring now to FIG. 2, a perspective view of a bad breath detection strip (100) is shown in accordance with an alternative embodiment of the present invention. The bad breath detection strip (100) includes a mouthpiece (102) at an end and a grip (104) at an opposing end. The mouthpiece (102) includes the indicating agent to detect the malodorous chemicals and compounds in the saliva of the user. The user may hold the grip (104) and place the mouthpiece (102) into their mouth. The saliva in the mouth wets the mouthpiece (102), and then user extracts the bad breath detection strip (100). The indicating agent may change color depending on the concentrations of malodorous compounds present in the saliva, as explained when referring to FIG. 1. After use, the bad breath detection strip (100) may be thrown away and replaced with a new strip.

Referring to FIG. 3, a bad breath detection disc (200) is shown in accordance with an alternative embodiment of the present invention. The bad breath detection disc (200) comprises a cylindrical body (202) with an elevated edge (204). The cylindrical body (202) may be covered with the indicating agent to detect the malodorous compounds present in the saliva of the user. The user may place the bad breath detection disc (200) in their mouth and then extract the disc after it is wet by the user's saliva. The indicating agent may change colors depending on the concentration of malodorous compounds present, as explained while referring to FIG. 1.

The bad breath detecting devices allow a person to quickly check their breath while they are without their toothbrush, to determine if they need gum or a mint to help improve the freshness of their breath until they are able to brush again. The person may easily carry one or more of the device in their bag or pocket, and then dispose of the used device after use.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A bad breath detection system comprising:
a bad breath detection device; and
a indicating agent on the bad breath detection device wherein when a user places the bad breath detection device in their mouth the indicating agent provides a visual cue depending on a concentration of malodorous compounds in a saliva sample of the user to indicate to the user a level of breath freshness.

2. The bad breath detection system according to claim 1, wherein the visual cue is a color change.

3. The bad breath detection system according to claim 2, where the color change goes from a light color to a dark color relative to the concentration of malodorous compounds in the saliva sample.

4. The bad breath detection system according to claim 2, where the color change indicates a specific concentration depending on a specific color of the indicating agent.

5. The bad breath detection system according to claim 1, wherein the bad breath detection device is a tube including an elongated body, a mouthpiece and an evacuation end, where the tube enables the user to exhale into the mouthpiece to determine the concentration of malodorous compounds in the saliva sample exhaled by the user.

6. The bad breath detection system according to claim 1, wherein the bad breath detection device is a strip, where an end of the strip is placed in the mouth of the user to determine the concentration of malodorous compounds.

7. The bad breath detection system according to claim 1, wherein the bad breath detection device is a disc, where the disc is placed in the mouth to determine the concentration of malodorous compounds.

8. The bad breath detection system according to claim 1, wherein the bad breath detection device is reusable.

9. The bad breath detection system according to claim 1, wherein the bad breath detection device is disposable.
